(19) United States
(12) Patent Application Publication
(10) Pub. No.: US 2003/0194790 A1
(43) Pub. Date: Oct. 16, 2003

(54) DNA sequences coding for ester-group-cleaving enzymes

(76) Inventors: Wolf-Dieter Deckwer, Braunschweig (DE); Rolf-Joachim Mueller, Braunschweig (DE); Joop van den Heuvel, Braunschweig (DE); Ilona Kleeberg, Braunschweig (DE); Ute Widow, Braunschweig (DE)

Correspondence Address: FROMMER LAWRENCE & HAUG, 745 FIFTH AVENUE- 10TH FL., NEW YORK, NY, 10151, US

(21) Appl. No.: 10/102,239

(22) Filed: Mar. 20, 2002

(51) Int. Cl. C12P 7/40; C12N 9/16; C12Q 1/68; C07H 21/04; C12P 21/02; C12N 5/06; C07K 16/40

(52) U.S. Cl. 435/136; 435/6; 435/69.1; 435/196; 435/320.1; 435/325; 536/23.2; 530/388.26

(57) ABSTRACT

The invention relates to DNA sequences coding for enzymes that cleave or hydrolyse ester groups, to the production of those enzymes by genetically recombinant microorganisms and to the use of those enzymes and microorganisms for the degradation of polymers containing ester groups, especially aliphatic-aromatic polyesters.

The invention relates to DNA sequences coding for enzymes that cleave or hydrolyse ester groups (hereinafter also EGCEs or EGCE-1 and EGCE-2), to the production of those enzymes by genetically recombinant microorganisms and to the use of those enzymes and microorganisms for the degradation of polymers containing ester groups.

Polymers and macromolecular materials that can be biologically degraded in a controlled manner are becoming increasingly important. A number of such products is already available on the market. Amongst those new types of product, polymers containing ester groups (for example polyesters, polyester urethanes, polyester amides) occupy a central role. Examples of biodegradable polyester-based plastics are poly(β-hydroxybuty-rate-co-β-hydroxyvalerate), poly(ε-caprolactone) and poly(buty-lenesuccinate).

Since, owing to their molecular size, polymers are unable to pass through the outer membrane of microbe cells, the first, and generally the rate-determining, step of degradation is molecular weight reduction (depolymerisation) by extracellular enzymes. Polyesters are potentially bio-degradable because the ester bonds fundamentally represent points of attack for such extracellular hydrolysing enzymes.

For aliphatic polyesters, there have long been studies on biological degradation by means of such hydrolysing enzymes (for example lipases, PHB-depolymerases) [1) Tokiwa et al., Polym. Mater. Sci. Eng. 62 (1990), 988-992] [2) Jendrossek et al., Appl. Microbiol. Biotechnol. 46 (1996), 451-463]. The material is incubated with an appropriate enzyme under suitable conditions and the degradation is determined by the formation of cleavage products in the surrounding medium or by the weight loss of the samples. In the case of natural polyhydroxyalkanoates, specifically isolated hydrolases (PHB-depolymerases) have generally been used for this, whereas, for the degradation of synthetic polyesters, commercial lipases etc. that have not been specifically isolated for the purpose have been used.

While many aliphatic polyesters have proved in principle to be open to biological attack, as is generally known aromatic polyesters (for example polyethylene terephthalate, polypropylene terephthalate, polybutylene terephthalate) are considered to be biologically resistant. In order to make use of the better processing and performance properties of aromatic structures in comparison with aliphatic polyesters, there have been developed in the last few years biologically degradable aliphatic-aromatic copolyesters, which are manufactured on an industrial scale.

By introducing aromatic components, however, the rate of biological degradation is significantly reduced [3) Müller et al., Polym. Degrad. Stab. 59 (1998), 203-208]. Thus, for example, Jun et al., [4) Jun et al., J. Environ. Polym. Degrad. 2(1) (1994), 9-18] come to the conclusion that copolyesters of PET and PCL are not significantly attacked by lipases (for example Pseudomonas-sp.-lipase).

The degradation of (especially) polyester amides using various conventional commercial lipases from a technical point of view has recently been described [5) WO 98/36086]. In that patent specification, the degradation of a copolyester of butanediol, terephthalate (40 mol %) and adipate (60 mol %) is also described. The degradation reactions that may possibly be suitable for technical applications were produced, for example, by the addition of 50 mg of enzyme (lipase from Candida antarctica) to 0.3-1.8 g of a polyester amide in sheet or plate form, the degradation rates achieved being in the region of 600 mg degradation/week. For the described degradation of an aliphatic-aromatic copolyester the enzyme has to be added in a concentration of 1% (in 100 ml of buffer) to a fine powder of the copolyester. Despite the considerably larger surface area due to the small particle size, only a degradation of 230 mg/week is thus achieved.

It has recently been possible to show that aliphatic-aromatic copolyesters can be degraded by microorganism strains from the group of the Actinomycetes [6) Kleeberg et al., Appl. Environ. Polym. Degrad. 64(5) (1998), 1731-1735]. It was found that biologically degradable polymers containing polyester groups, especially also aliphatic-aromatic copolyesters, are depolymerised and broken down into lower-molecular-weight fragments with an extraordinarily high degradation rate by the extracellular enzyme from the Actinomycetes strain Thermomonospora fusca (DSM 43793) on its own or in admixture with other enzymes. Using methods relying on the use of that new kind of enzyme, it is possible to achieve degradation rates that are distinctly superior to those of hitherto-known systems and that render possible the technical exploitation of the enzymatic treatment of polymers containing ester groups. That applies especially to aliphatic-aromatic copolyesters and polyester blends, which are of great economic importance.

The ester-group-cleaving enzymes (EGCEs) (which form the subject-matter of pending Patent Application DE 199 47 286.6 of the Applicant) can be isolated by culturing the microorganism Thermomonospora fusca in a suitable nutrient medium under thermophilic conditions, where appropriate in the presence of an inductor. Since the production of the enzymes is subject to feedback regulation, they cannot be produced in a concentration exceeding a certain maximum concentration. It would therefore be desirable if the production of the, ester-group-cleaving enzymes (EGCEs) could be carried out using genetic engineering techniques.

The object of the invention is therefore the provision of DNA sequences that code for the new thermostable ester-group-cleaving enzymes (EGCEs) from Thermomonospora fusca, the creation of microorganisms genetically modified therewith and the use of the expression or partial expression products coded by the DNA sequences and having ester-group-cleaving activity, or of the genetically modified microorganisms, for the degradation of polymers containing ester groups.

The invention accordingly provides a DNA sequence (Seq ID No 1) that is the regulon of the ester-group-cleaving enzymes and that comprises a DNA sequence selected from among:

(a) the following DNA sequence:

ACGTGTCCTC TGCACTGCAA TCCCCGCTTC CCAGCGCATC ACCGGCCCCG GCGCGGGCCG
GACGCGGCGG GGTGCTCCCC GCGCCCGGCT GGGTTGTGGG TGCGGTCTGC ACGGTGTTCG
CCGTCGCGGG CCTCACCCTG GTCCCGCCGT GGCTGGGCCT GGGATGGGAC GAGGTCGTCT
ACGTCAGCCA GTACGATCCC CGCAATCCCG CCGCGTTCTT CAGCGCGCCC CCGTCGCGCG
GGGTGTCGCT GCTGGCCGCG CCGGTGGTGC TCGTCACCGA CTCGGTGGTG GCGCTTCGGG
TGTGGCTGGC TGCGGCCGCG GCCGTGGCCA TGGGCGCGGC GTTCTGGCCG TGGCTGCGGC
TGTATCCGCG CAGCGGGGTG GTGCCGCTCG CAGCCTTCGG GTATGCGAGC CTGTGGGTCA
GCTTGTTCTA CGCGGCCGCG GCGATGCCCA ACCATTTCAC GGCGATGGCC GCGGTGGGCG
CGGTCGGCTG GTTCCTGGTC GCGGTCCGCG AACCCGCGTC CCGGTCTGCG CTGGCCGGGC
TTGCAGCCAT GCTGGCGGTG GCCGGGCTGA TGCGGCCCAG TGACGCGTTC TGGTTGACCG
CGCCTTTGGG GCTGGCCGGA CTTGTCGTGC CGTCGTGGCG GCGGGTACCG CTGCTCGCGG
CCGTGGCCGG CGGCGGCCTG GCCGGTGTGG CACCGTGGCT GGTGGAAGCG GAGCTCTCCT
ACGGGGGCGT GCTCTCCCGG CTGGCTCGGG CCTCCGAGAT CCAGGGCGGG ACCGGGTGGA
CCCTGGCTGT GGGGTACGTG GTCACCGCGT TGGACGGGCC GCTGCTGTGC CGCCCCTGCA
CCGAGGACCT GGTGCGCTGG CCTGCCCTGC TCTGGCCGAT CGCTTTGGTC GTACTGGTGG
TGGGCGGGAT TGTCGGCGCT CACCGGGCGG GACGTCCCGC TCTGGGGTGG CTGCCTGTGG
TGGTCGCCGG GTCGCTCGCG TTCACCTACC TGTTCCTCGT CTCCTACACG GCGCCGCGTT
TCCTCCAGCC CACGTACGCG CTGCTCATGC TGCCCGCTGC GGCAGGACTG CACGATGCGT
GGACGGCGAC CCGGCCCCGG CTGCGTCCCG TGCTGGGTGC CGGGTTGGCC GTGGCGATCC
TCGGACATCT CCTCATCCAG GGGGCGATCC TCACCCACTG CGTGACCGTC CACACGGCGG
CCCGGGAGAA CTATGCCCGG CTCGCCGAGG AGCTGCATCC GGCCGGGCTA CGCCCGCCGT
GCGTGCTCAC CGGGGACGAG GCCATTCCCA TCGCCTACTA CGCGGGGTGC GCCTCGGCGG
CGGCGTCCGG CAACAACACC ACGCACACCC TGGAGGAGCT GCTCGCGGTG AGCCGGACCG
TACCGTTCGG GCTGCTCGCC AAGGAGGACG GTCCGCCCGA GTGGGCCGCT GACTGGGACG
GGCTGCCCGT GGGTCCCGCC GACGACCCGT GGTCGTGGGT GGTGTACCTC CCGCCGTGGA
GCCCGCTGTC CATTCCGGAA TGAGTCCGAG CGGGTATTCT CGCTACCTAT TTCAGCCCCG
GAGTCAGGAT TCCGGGCTTT TTCTCTGTCC CACCCCACCC CCACATTTAT GGACATTTCC
TCGCAAAACA CACTATTTGA CCTGTGGTTT GGCGAGACAC TGGTGATTTC ACGGATGCCA
TCCGGCTCCC CCATGCCGAA TAGTGACGTT GCGGTTAAGA CACAGAACCG GTTACCGCCG
GATCTCCTTA CCGCAACGTT GTGAGCGGCC TACCGCAATG GCTGACCACG ACGAGGCAGA
CCCTCGCCCA CCAGTGCCTG CCGCATCGGC CCCCCGCTGC GACGGTCACG CCCGGCTTCG
GACTCTCGGG GACGGCGCCC CGGTGGGCAT GGACCGTTCA GTGTCCCCAC GGTGAACGGC
CCACCATCCC CCGCACATCC GGTCTGCCCC TACCGTGGCC AGTGCCGCTC CTCCCTCCGT
CCACGGGCGA CCCTCCGCTT TCGCCCTTAC ACGAAGAGGA TGTGCAATGG CTGTGATGAC
CCCCCGCCGG GAGCGCTCTT CCCTGCTCTC CCGAGCTCTG CAAGTGACGG CTGCGGCTGC
CACAGCGCTT GTGACCGCGG TCAGCCTGGC CGCCCCCGCT CATGCCGCCA ACCCCTACGA
GCGCGGCCCC AACCCGACCG ACGCCCTGCT CGAAGCCAGC AGCGGCCCCT TCTCCGTCAG
CGAGGAGAAC GTCTCCCGGT TGAGCGCCAG CGGCTTCGGC GGCGGCACCA TCTACTACCC
GCGGGAGAAC AACACCTACG GTGCGGTGGC GATCTCCCCC GGCTACACGG GCACTGAGGC
TTCCATCGCC TGGCTGGGCG AGCGCATCGC CTCCCACGGC TTCGTCGTCA TCACCATCGA
CACCATCACC ACCCTCGACC AGCCGGACAG CCGGGCAGAG CAGCTCAACG CCGCGCTGAA
CCACATGATC AACCGGGCGT CCTCCACGGT GCGCAGCCGG ATCGACAGCA GCCGACTGGC
GGTCATGGGC CACTCCATGG GCGGCGGCGG CACCCTGCGT CTGGCCTCCC AGCGTCCCGA
CCTGAAGGCC GCCATCCCGC TCACCCCGTG GCACCTCPAC AAGAACTGGA GCAGCGTCAC
CGTGCCGACG CTGATCATCG GGGCCGACCT CGACACAATC GCGCCGGTCG CCACGCACGC
GAAACCGTTC TACAACAGCC TGCCGAGCTC CATCAGCAAG GCCTACCTGG AGCTGGACGG
CGCAACCCAC TTCGCCCCGA ACATCCCCAA CAAGATCATC GGCAAGTACA GCGTCGCCTG
GCTCAAGCGG TTCGTCGACA ACGACACCCG CTACACCCAG TTCCTCTGCC CCGGACCGCG
CGACGGACTC TTCGGCGAGG TCGAAGAGTA CCGCTCCACC TGCCCGTTCT AGGAAGAGAA
CACGACGAGT CTTTCCTCCC CATTCTTTCG GTGGCGGTCA CTGCGGTGGC CGCCACCGGC
CGTTTTGTCC CCCCTTTTCA TTCGAAAAAT GCGACAAACC ACCCTTTTTG CCCCATCGCA
CCCCCGATAC CGAACGAAGT TCGGGTACAA CACTGGTGGT TTTACGGATG CATGATCACT
GTGACTTGCC CCATAGTGGC AACGCAGTCG AGATAAGGAG CGCATAAACC CCAAACCTCC
TTACCTCCGC CTGCTGAGCG GCTCGTTGAC CGCACGTGGC CGGGCACACC GGCTTCGCCT
ACCGGTCGCA CGCGCCGTGC CTTCTCCACC CCCGCGGCGG AAAGGCGCGG CGCTCGCGGG
CTGGACCGTT CGGACCCCAC GCGAACGGCC CGGAACCCAT GGCACCCCCG CGTCCGGGAG
GCAAGCGCCG CGTGCCTACC GCCAACGGCG CCGCTCACCT CCAGTGGCGA GGCGGGAGTC
CGGGTCCACG TCCATGCATG CCCCCGCATG CGGCGCGGCC CGGCCCTGCA CAGAACCGAA
GAGGACGTGC AATGGCTGTG ATGACCCCCC GCCGGGAGCG CTCTTCCCTG CTCTCCCGGG
CACTGCGCTT CACCGCCGCG GCTGCCACAG CGCTTGTGAC CGCGGTCAGC CTGGCCGCCC
CCGCTCATGC CGCCAACCCC TACGAGCGCG GCCCCAACCC GACCGACGCC CTGCTCGAAG
CCCGCAGCGG CCCCTTCTCC GTGAGTGAAG AACGGGCCTC CCGCTTCGGT GCTGACGGTT
TCGGCGGCGG CACCATCTAC TACCCGCGGG AGAACAACAC CTACGGTGCC GTGGCGATCT
CCCCCGGCTA CACCGGCACC CAGGCCTCTG TCGCCTGGCT GGGCAAGCGC ATCGCCTCCC
ACGGCTTCGT CGTCATCACC ATCGACAGCA ACACCACCCT CGACCAGCCG GACAGCCGGG
CCCGCCAGCT CAACGCCGCG CTGGACTACA TGATCAACGA CGCCTCGTCC GCGGTGCGCA
GCCGGATCGA CAGCAGCCGA CTGGCGGTCA TGGGCCACTC CATGGGCGGC GGCGGCAGCC
TCCGTCTGGC CTCCCAGCGT CCCGACCTGA AGGCCGCCAT CCCGCTCACC CCGTGGCACC
TCAACAAGAA CTGGAGCAGT GTGCGGGTTC CCACCCTCAT CATCGGTGCT GACCTGGACA
GCATCGCTCC GGTCCTCACC CACGCCCGGC CCTTCTACAA CAGCCTCCCG ACCTCGATCA
GCAAGGCCTA CCTGGAGCTG GACGGCGCAA CCCACTTCGC CCCGAACATC CCCAACAAGA
TCATCGGCAA GTACAGCGTC GCCTGGCTCA AGCGGTTCGT CGACAACGAC ACCCGCTACA
CCCAGTTCCT CTGCCCCGGA CCGCGCGACG GACTCTTCGG CGAGGTCGAA GAGTACCGCT
CCACCTGCCC CTTCTAGGCG GTAGGGTCCC GGAGCCAGTC AGCAAGATCT CCTTCCCGGT
GGTTGATACT G

or the complementary strand thereof,

(b) DNA sequences that hybridise under stringent conditions to protein-encoding regions of a DNA sequence according to (a) or to fragments of such a DNA sequence,

(c) DNA sequences that hybridise to DNA sequences according to (a) or (b) owing to degeneracy of the genetic code,

(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

The DNA has 4451 bases in the single strand, these being: 648 A; 1684 C; 1377 G: 742 T.

That DNA sequence permits the production of the EGCEs in genetically recombinant microorganisms in which a further increase in productivity is not rendered impossible by feedback regulation. The genetically recombinant microorganisms and the ester-group-cleaving enzymes produced by them can be used for the biological degradation of polymers containing ester groups.

The invention furthermore provides a DNA sequence (Seq ID No 2) that codes for the ester-group-cleaving enzyme EGCE-1 and that comprises a DNA sequence selected from among:

(a) the following DNA sequence:

ATGGCTGTGA TGACCCCCCG GCGGGAGCGC TCTTCCCTGC TCTCCCGAGC TCTGCAAGTG
ACGGCTGCGG CTGCCACAGC GCTTGTGACC GCGGTCAGCC TGGCCGCCCC CGCTCATGCC
GCCAACCCCT ACGAGCGCGG CCCCAACCCG ACCGACGCCC TGCTCGAAGC CAGCAGCGGC
CCCTTCTCCG TCAGCGAGGA GAACGTCTCC CGGTTGAGCG CCAGCGGCTT CGGCGGCGGC
ACCATCTACT ACCCGCGGGA GAACAACACC TACGGTGCGG TGGCGATCTC CCCCGGCTAC
ACCGGCACTG AGGCTTCCAT CGCCTGGCTG GGCGAGCGCA TCGCCTCCCA CGGCTTCGTC
GTCATCACCA TCGACACCAT CACCACCCTC GACCAGCCGG ACAGCCGGGC AGAGCAGCTC
AACGCCGCGC TGAACCACAT GATCAACCGG GCGTCCTCCA CGGTGCGCAG CCGGATCGAC
AGCAGCCGAC TGGCGGTCAT GGGCCACTCC ATGGGCGGCG GCGGCACCCT GCGTCTGGCC
TCCCAGCGTC CCGACCTGAA GGCCGCCATC CCGCTCACCC CGTGGCACCT CAACAAGAAC
TGGAGCAGCG TCACCGTGCC GACGCTGATC ATCGGGGCCG ACCTCGACAC AATCGCGCCG
GTCGCCACGC ACGCGAAACC GTTCTACAAC AGCCTGCCGA GCTCCATCAG CAAGGCCTAC
CTGGAGCTGG ACGGCGCAAC CCACTTCGCC CCGAACATCC CCAACAAGAT CATCGGCAAG
TACAGCGTCG CCTGGCTCAA GCGGTTCGTC GACAACGACA CCCGCTACAC CCAGTTCCTC
TGCCCCGGAC CGCGCGACGG ACTCTTCGGC GAGGTCGAAG AGTACCGCTC CACCTGCCCG
TTC

or the complementary strand thereof,

(b) DNA sequences that hybridise under stringent conditions to protein-encoding regions of a DNA sequence according to (a) or to fragments of such a DNA sequence,

(c) DNA sequences that hybridise to DNA sequences according to (a) or (b) owing to degeneracy of the genetic code,

(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

That DNA has 903 bases in the single strand, these being: 163 A; 364 C; 249 G; 127 T.

They are bases 2027 to 2929 of the EGCE regulon of Seq ID No 1.

The invention furthermore provides a DNA sequence (Seq ID No 3) that codes for the ester-group-cleaving enzyme EGCE-2 and that comprises a DNA sequence selected from among:

(a) the following DNA sequence:

ATGGCACCCC CGCGTCCGGG AGGCAAGCGC CGCGTGCCTA CCGCCAACGG CGCCGCTCAC
CTCCAGTGGC GAGGCGGGAG TCCGGGTCCA CGTCCATGCA TGCCCCCGCA TGCGGCGCGG
CCCGGCCCTG CACAGAACCG AAGAGGACGT GCAATGGCTG TGATGACCCC CCGCCGGGAG
CGCTCTTCCC TGCTCTCCCG GGCACTGCGC TTCACCGCCG CGGCTGCCAC AGCGCTTGTG
ACCGCGGTCA GCCTGGCCGC CCCCGCTCAT GCCGCCAACC CCTACGAGCG CGGCCCCAAC
CCGACCGACG CCCTGCTCGA AGCCCGCAGC GGCCCCTTCT CCGTGAGTGA AGAACGGGCC
TCCCGCTTCG GTGCTGACGG TTTCGGCGGC GGCACCATCT ACTACCCGCG GGAGAACAAC
ACCTACGGTG CCGTGGCGAT CTCCCCCGGC TACACCGGCA CCCAGGCCTC TGTCGCCTGG
CTGGGCAAGC GCATCGCCTC CCACGGCTTC GTCGTGATCA CCATCGACAC CAACACCACC
CTCGACCAGC CGGACAGCCG GGCCCGCCAG CTCAACGCCG CGCTGGACTA CATGATCAAC
GACGCCTCGT CCGCGGTGCG CAGCCGGATC GACAGCAGCC GACTGGCGGT CATGGGCCAC
TCCATGGGCG GCGGCGGCAG CCTGCGTCTG GCCTCCCAGC GTCCCGACCT GAAGGCCGCC
ATCCCGCTCA CCCCGTGGCA CCTCAACAAG AACTGGAGCA GTGTGCGGGT TCCCACCCTC
ATCATCGGTG CTGACCTGGA CACCATCGCT CCGGTCCTCA CCCACGCCCG GCCCTTCTAC
AACAGCCTCC CGACCTCGAT CAGCAAGGCC TACCTGGAGC TGGACGGCGC AACCCACTTC
GCCCCGAACA TCCCCAACAA GATCATCGGC AAGTACAGCG TCGCCTGGCT CAAGCGGTTC
GTCGACAACG ACACCCGCTA CACCCAGTTC CTCTGCCCCG GACCGCGCGA CGGACTCTTC
GGCGAGGTCG AAGAGTACCG CTCCACCTGC CCCTTC

or the complementary strand thereof,

(b) DNA sequences that hybridise under stringent conditions to protein-encoding regions of a DNA sequence according to (a) or to fragments of such a DNA sequence,

(c) DNA sequences that hybridise to DNA sequences according to (a) or (b) owing to degeneracy of the genetic code,

(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

That DNA has 1056 bases in the single strand, these being: 177 A; 437 C; 293 G; 149 T.

They are bases 3339 to 4394 of the EGCE regulon of Seq ID No 1.

The invention further relates to the expressed pre-enzyme of the ester-group-cleaving enzyme 1 (preEGCE-1) having an amino acid sequence that comprises an amino acid sequence (Seq ID No 4) selected from among

(a) the following amino acid sequence:

MAVMTPRRER SSLLSRALQV TAAAATALVT AVSLAAPAHA ANPYERGPNP TDALLEASSG
PFSVSEENVS RLSASGFGGG TIYYPRENNT YGAVAISPGY TGTEASIAWL GERIASHGFV
VITIDTITTL DQPDSRAEQL NAALNHMINR ASSTVRSRID SSRLAVMGHS MGGGGTLRLA
SQRPDLKAAI PLTPWHLNKN WSSVTVPTLI IGADLDTIAP VATHAKPFYN SLPSSISKAY
LELDGATHFA PNIPNKIIGK YSVAWLKRFV DNDTRYTQFL CPGPRDGLFG EVEEYRSTCP
F

(b) an amino acid sequence 'that is an allelic isofunctional variant of the amino acid sequence defined in (a),

(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,

(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

PreEGCE-1 has 301 amino acids and a molecular weight of 32182 daltons. The translation start point is base 1 of the DNA sequence of Seq ID No 2.

The invention further relates to the expressed pre-enzyme of the ester-group-cleaving enzyme 2 (preEGCE-2) having an amino acid sequence that comprises an amino acid sequence (Seq ID No 5) selected from among

(a) the following amino acid sequence:

MAPPRPGGKR RVPTANGAAH LQWRGGSPGP RPCMPPHAAR PGPAQNRRGR AMAVMTPRRE
RSSLLSRALR FTAAAATALV TAVSLAAPAH AANPYERGPN PTDALLEARS GPFSVSEERA
SRFGADGFGG GTIYYPRENN TYGAVAISPG YTGTQASVAW LGKRIASHGF VVITIDTNTT
LDQPDSRARQ LNAALDYMIN DASSAVRSRI DSSRLAVMGH SMGGGGSLRL ASQRPDLKAA
IPLTPWHLNK NWSSVRVPTL IIGADLDTIA PVLTHARPFY NSLPTSISKA YLELDGATHF
APNIPNKIIG KYSVAWLKRF VDNDTRYTQF LCPGPRDGLF GEVEEYRSTC PF

(b) an amino acid sequence that is an allelic isofunctional variant of the amino acid sequence defined in (a),

(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,

(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

PreEGCE-2 has 352 amino acids and a molecular weight of 37798 D. The translation start point is base 1 of the DNA sequence of Seq ID No 3.

The invention further relates to the expressed mature enzyme of the ester-group-cleaving enzyme 1 (matureEGCE-1) having an amino acid sequence that comprises an amino acid sequence (Seq ID No 6) selected from among

(a) the following amino acid sequence:

AANPYERGPN PTDALLEASS GPFSVSEENV SRLSASGFGG GTIYYPRENN TYGAVAISPG
YTGTEASIAW LGERIASHGF VVITIDTITT LDQPDSRAEQ LNAALNHMIN RASSTVRSRI
DSSRLAVMGH SMGGGGTLRL ASQRPDLKAA IPLTPWHLNK NWSSVTVPTL IIGADLDTIA
PVATHAKPFY NSLPSSISKA YLELDGATHF APNTPNKIIG KYSVAWLKRF VDNDTRYTQF
LCPGPRDGLF GEVEEYRSTC PF

(b) an amino acid sequence that is an allelic isofunctional variant of the amino acid sequence defined in (a),

(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,

(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

MatureEGCE-1 has 262 amino acids and a molecular weight of 28214 daltons. The translation start point is base 118 of the DNA sequence of Seq ID No 2.

The invention further relates to the expressed mature enzyme of the ester-group-cleaving enzyme 2 (matureEGCE-2) having an amino acid sequence that comprises an amino acid sequence (Seq ID No 7) selected from among

(a) the following amino acid sequence:

AANPYERGPN PTDALLEARS GPFSVSEERA SRFGADGFGG GTIYYPRENN TYGAVAISPG
YTGTQASVAW LGKRIASHGF VVITIDTNTT LDQPDSRARQ LNAALDYMIN DASSAVRSRI
DSSRLAVMGH SMGGGGSLRL ASQRPDLKAA IPLTPWHLNK NWSSVRVPTL IIGADLDTIA
PVLTHARPFY NSLPTSISKA YLELDGATHF APNIPNKIIG KYSVAWLKRF VDNDTRYTQF
LCPGPRDGLF GEVEEYRSTC PE

(b) an amino acid sequence that is an allelic isofunctional variant of the amino acid sequence defined in(a),

(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,

(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

MatureEGCE-2 has 262 amino acids and a molecular weight of 28422 daltons. The translation start point is base 271 of the DNA sequence of Seq ID No 3.

The invention further relates to recombinant expression vectors that contain a DNA sequence according to the invention or fragments thereof, to prokaryotic or eukaryotic cells, for example E. coli or P. pastoris, that have been transformed or transfected with a DNA sequence according to the invention or fragments thereof or with a recombinant expression vector according to the invention, and to the expression products or partial expression products of the DNA-sequences according to the invention or of the recombinant expression vectors.

The invention further relates to a process for the production of the expression products or partial expression products according to the invention, in which cells according to the invention are cultured in a suitable culture medium and the expression products or partial expression products are isolated from the cells and/or from the culture medium.

The invention further relates to polyclonal or monoclonal antibodies to expression products according to the invention or to amino acid sequences according to the invention.

The invention further relates to the use of the expression products or partial expression products according to the invention for the biological degradation of polymers containing ester groups, especially aliphatic-aromatic polyesters.

The subsidiary claims relate to advantageous and/or preferred embodiments of the invention.

The invention is described in detail below, without limitation and with reference to illustrative embodiments.

The amino acid sequence of the ester-group-cleaving enzyme (EGCE) (which forms the subject-matter of pending Patent Application DE 199 47 286.6 of the Applicant) was converted with the aid of the genetic code into a degenerate DNA sequence. Based on that sequence, oligonucleotides were constructed for PCR amplification.

A new process (cf. Example 1) was furthermore developed for the rapid and orderly purification of genomic DNA from Thermomonospora fusca and was used to analyse the genomic DNA from T. fusca by means of Southern blot analysis.

The PCR oligonucleotides were used to amplify a 720 bp DNA fragment that codes for 90% of the secreted enzyme. That fragment was used for the identification and cloning of a genomic fragment having 8 kbp from a genomic bank of T. fusca DSM 47393 which was produced for this invention.

The DNA sequence was characterised by means of PCR sequencing, which was specifically modified according to the information given by the manufacturer (ABI Prism Bigdye Terminator Kit, Applied Biosystems Perkin Elmer, Weiterstadt, Langen) for the sequencing of that fragment because of the extremely high GC content. Analysis of the DNA sequence revealed the presence of two genes that code for ester-group-cleaving enzymes. Both enzymes (EGCE-1 and EGCE-2) are very homologous (92%) and have a pre-sequence that is necessary for transport in the medium. The coding sequence of those genes was cloned in expression vectors for E. coli and Pichia pastoris and the activity of the recombinant enzyme was demonstrated. Expression could be achieved if the native transport signals for the genes of the ester-group-cleaving enzymes were replaced by E. coli presequences (such as, for example, OmpA, PhoA or LamB).

The invention permits:

simple production of the EGC enzymes with known host strains,

substrate-independent induction of enzyme secretion and production,

mesophilic production of the enzymes,

the achievement of high activity,

the possibility of metabolic engineering of the microorganisms by means of site-directed mutagenesis or directive evolution by means of PCR mutagenesis for improving the ester-group-cleaving activity of the enzymes.

By using genetically recombinant microorganisms that over-produce EGC enzymes in situ, polyester degradation is possible on a large scale.

Surprisingly, the ester-group-cleaving activity in genetically recombinant microorganisms is higher than that of T. fusca and is not subject to substrate-dependent induction of the gene expression.

### EXAMPLES

### Example 1

Working-up of the Chromosomal DNA of Thermomonospora fusca

1 g of mycelium is thawed and homogenised in 5 ml of TE (10 mM Tris-HCl, 1 m EDTA pH 8.0). 10 mg of lysozyme are added and the solution is mixed for 30 seconds. Incubation is then carried out at 37° C. for from 30 to 60 minutes. 1.2 ml of 0.5 M EDTA and 13 μl of Pronase (20 mg/ml) are then added and the whole is mixed and incubated for a further 5 minutes. After the addition of 0.7% sodium dodecyl sulphate (0.43 ml of a 10% strength SDS stock solution), the whole is mixed and incubated at 37° C. for from 1 to 2 hours. Incubation can be brought to an end when the lysate becomes “clear”. The extract is purified by means of phenol extraction with 10 ml of Rotiphenol/TE, pH 7.5 (Roth, Art. 0038.2). The clear supernatant is then removed with a Pasteur pipette and transferred to a new vessel. The procedure is repeated once, whereupon extraction is carried out twice with 10 ml of chloroform each time. The protein-free supernatant is treated for from 30 to 60 minutes at 37° C. (or overnight at 4° C.) with 4 μl/ml of a stock solution RNAse having a concentration of 10 mg/ml. The volume is made up to 5 ml with TE, and 500 μl of 3 M NaCl and 3.5 ml of isopropanol are added. The DNA precipitation is carried out on ice or over-night at 4° C. The DNA pellet is centrifuged at 4000 g for 10 minutes and is then washed with 70% ethanol. After drying in air, the DNA can be taken up in 500 μl of TE and stored at −20° C. Up to 1 mg of DNA is obtained from 1 g of frozen mycelium.

### Example 2

Nested PCR of the chromosomal DNA
with degenerate oligonucleotides

| Reaction A | |
|---|---|
| 0.5 μg T.-f.-DNA | x μl |
| 10× PCR-buffer | 5 μl |
| 2 mM dNTPs | 5 μl |
| Primer 1F 100 pmol/μl | 1 μl |
| Primer 2R 100 pmol/μl | 1 μl |
| Taq-polymerase (2-5 U) | 0.5 μl |
| MilliQ-water | y μl |
| final volume | 50 μl |
| PCR programme: | |
| denaturing for 5 min at 95° C., 30 cycles of 1 minute at 95° C., | |
| 1 minute at 55° C. and 2 minutes at 72° C. Extension 5 min at | |
| 72° C. After end of programme, store at 4° C. | |
| Reaction B | |
| Product of Reaction A | 10.0 μl |
| 10× PCR-buffer | 4.0 μl |
| 2 mM dNTPs | 5.0 μl |
| Primer 3F 100 pmol/μl | 1.0 μl |
| Primer 4R 100 pmol/μl | 1.0 μl |
| Taq-polymerase (2-5 U) | 0.5 μl |
| MilliQ-water | 28.5 μl |
| final volume | 50.0 μl |

The same PCR programme as that in Reaction A was applied.

Primer 1F:
AGA GGA GAA TTC AAY CCN TAY GAR MGI GGN AAY CC-3′
Primer 2R:
AGA GGA TCT AGA GGN GGR CAN ARR AAY TGN GTR TA-3′
Primer 3F:
AAY GCN GCN YTN AAY CAY ATG AT-3′
Primer 4R:
YTT RTT NGG DAT RTT NGG NGC-3′

Reaction A yielded a fragment having 700 bp which was cloned via the EcoRI and XbaI cleavage sites directly into pUC19 using standard protocols [7) Sambrook et al., Molecular Cloning, a Laboratory Manual, Cold Spring Harbor Laboratory Press 1989].

### Example 3

Cloning of the Regulon of the EGC Enzymes from T. fusca

Chromosomal DNA from T. fusca was cleaved with HindIII, BamHI, PstI and EcoRI. The DNA fragments were separated according to size by means of agarose gel electrophoresis. The separated fragments were then transferred to nylon membranes (Amersham, Hybond-N) and hybridised with the 700 bp fragment (Example 2) obtained by EcoR1-Xba1-restriction. Upon Southern blotting [7) Sambrook et al., Molecular Cloning, a Laboratory Manual, Cold Spring Harbor Laboratory Press 1989], specific hybridisation signals were obtained. Those Southern blots exhibited individual bands corresponding to fragment lengths of 13 Kb for Hind III, 8 Kb for BamHI, 7.2 Kb for PstI and 15 Kb for EcoRI. An amplified chromosomal 6 to 10 Kb DNA bank of T. fusca in pUC19 was produced and analysed using the 700 bp fragment obtained by EcoR1-Xba1-restriction as a probe. Positive clones were isolated and characterised by Southern blot hybridisation. From the master clone, T.f.-Ilo 20, subclones were produced for sequencing. The DNA sequence of the regulon is given in claim 1.

### Example 4

Cloning and Expression of the EGCE Genes in E. coli

The genes that code for EGCE-1 and EGCE-2 were subjected to mutagenesis and amplified. The PCR primers TFEGSE1F1 and TFEGSE1R1 were used to amplify preEGCE-1. The PCR primer pair TFEGSE1F2 and TFEGSE1R1 was used to amplify EGCE1.

| | |
|---|---|
| DNA fragment from | 2.0 μl |
| clone T.f.-Ilo 20 | |
| 10× PCR-buffer | 5.0 μl |
| 2 mM dNTPs | 8.0 μl |
| Primer 1F1 100 pmol/μl | 2.0 μl |
| Primer 1R1 100 pmol/μl | 2.0 μl |
| VENT-polymerase (2-5 U) | 0.5 μl |
| MilliQ-water | 30.5 μl |
| final volume | 50.0 μl |

| | |
|---|---|
| TFEGSE1F1: | AGA GGA GAA TTC CAT ATG GCT GTG ATGT ACC CCC GC-3′ |
| TFEGSE1R1: | AGA GGA TCC TCT AGA CTA GAA CGG GCA GGT GGA GCG-3′ |
| TFEGSE1F2: | AGA GGA GAA TTC CAT ATG GCC AAC CCC TAC GAG CGC GGC-3′ |
| TFEGSE2F1: | AGA GGA GAA TTC CAT ATG GCT GTG ATGT ACC CCC GC-3′ |
| TFEGSE2F2: | AGA GGA GAA TTC CAT ATG GCT GTG ATGT ACC CCC GC-3′ |
| TFEGSE2R2: | AGA GGA TCC TCT AGA CTA GAA GGG GCA GGT GGA GCG-3′ |

### Example 5

Expression of the Recombinant EGC Enzymes in Active Form

Strain Top10 (E. coli K12) was transformed with pCYTEXP1-OmpA-EGCE-26, plated out onto polyester/LB/Amp tester plates and incubated overnight at 30° C. (repression of gene expression)

Further incubation was then carried out for 24 hours at 40° C. (induction of gene expression). The secreted EGC enzymes were active and caused large “clear zones” around the colonies on the turbid agar plates. The “clear zones” which the strain obtained by recombination produces are distinctly larger than those obtained with the T. fusca strain.

1. A DNA sequence that comprises a DNA sequence selected from among:
(a) the following DNA sequence:
ACGTGTCCTC TGCACTGCAA TCCCCGCTTC CCAGCGCATC ACCGGCCCCG GCGCGGGCCG
GACGCGGCGG GGTGCTCCCC GCGCCCGGCT GGGTTGTGGG TGCGGTCTGC ACGGTGTTCG
CCGTCGCGGG CGTCACCCTG GTCCCGCCGT GGCTGGGCCT GGGATGGGAC GAGGTCGTCT
ACGTCAGCCA GTACGATCCC CGCAATCCCG CCGCGTTCTT CAGCGCGCCC CGGTCGCGCG
GGGTGTCGCT GCTGGCCGCG CCGGTGGTGC TCGTCACCGA CTCGGTGGTG GCGCTTCGGG
TGTGGCTGGC TGCGGCCGCG GCCGTGGCCA TGGGCGCGGC GTTCTGGCCG TGGCTGCGGC
TGTATCCGCG CAGCGGGGTG GTGCCGCTCG CAGCCTTCGG GTATGCGAGC CTGTGGGTCA
GCTTGTTCTA CGCGGCCGCG GCGATGCCCA ACCATTTCAC GGCGATGGCC GCGGTGGGCG
CGGTCGGCTG GTTCCTGGTC GCGGTCCGCG AACCCGCGTC CCGGTCTGCG CTGGCCGGGC
TTGCAGCCAT GCTGGCGGTG GCCGGGCTGA TGCGGCCCAG TGACGCGTTC TGGTTGACCG
CGCCTTTGGG GCTGGCCGGA CTTGTCGTGC CGTGGTGGCG GCGGGTACCG CTGCTCGCGG
CCGTGGCCGG CGGCGGCCTG GCCGGTGTGG CACCGTGGCT GGTGGAAGCG GAGCTCTCCT
ACGGGGGCGT GCTCTCCCGG CTGGCTCGGG CCTCCGAGAT CCAGGGCGGG ACCGGGTGGA
CCCTGGCTGT GGGGTACGTG GTCACCGCGT TGGACGGGCC GCTGCTGTGC CGCCCCTGCA
CCGAGGACCT GGTGCGCTGG CCTGCCCTCC TCTGGCCGAT CGCTTTGGTC GTACTGGTGG
TGGGCGGGAT TGTCGGCGCT CACCGGGCGG GACGTCCCGC TCTGGGGTGG CTGCCTGTGG
TGGTCGCCGG GTCGCTCGCG TTCACCTACC TGTTCCTCGT CTCCTACACG GCGCCGCGTT
TCCTCCAGCC CACGTACGCG CTGCTCATGC TGCCCGCTGC GGCAGGACTG CACGATGCGT
GGACGGCGAC CCGGCCCCGG CTGCGTCCCG TGCTGGGTGC CGGGTTGGCC GTGGCGATCC
TCGGACATCT CCTCATCCAG GGGGCGATCC TCACCCACTG GGTCACCGTC CACACGGCGG
CCCGGGAGAA CTATGCCCGG CTCGCCGAGG AGCTGCATGC GGCCGGGCTA CGCCCGCCGT
GCGTGCTCAC CGGGGACGAG GCCATTCCCA TCGCCTACTA CGCGGGGTGC GCCTCGGCGG
CGGCGTCCGG CAACAACACC ACGCACACCC TGGAGGAGCT GCTCGCGGTG AGCCGGACCG
TACCGTTCGG GCTGCTCGCC AAGGAGGACG GTCCGCCCGA GTGGGCCGCT GACTGGGACG
CGCTGCCCGT GGGTCCCGCC GACGACCCGT GGTCGTGGGT GGTGTACCTC CCGCCGTGGA
GCCCGCTGTC CATTCCGGAA TGAGTCCGAG CGGGTATTCT CGCTACCTAT TTCAGGCCCG
GAGTCAGGAT TCCGGGCTTT TTCTCTGTCC CACCCCACCC CCACATTTAT GGACATTTCC
TCGCAAAACA CACTATTTGA CCTGTGGTTT GGCGAGACAC TGGTGATTTC ACGGATGCCA
TCCGGCTCCC CCATGCCGAA TAGTGACGTT GCGGTTAAGA CACAGAACCG GTTACCGGCG
GATCTCCTTA CCGCAACGTT GTGAGCGGCC TACCGCAATG GCTGACCACG ACGAGGCAGA
CCCTCGCCCA CGAGTGCCTG CCGCATCGGC CCCCCGCTGC GACGGTCACG CCCGGCTTCG
GACTCTCGGG GACGGCGCCC CGGTGGGCAT CGACCGTTCA GTGTCCCCAC GGTGAACGGC
CCACCATCCC CCGCACATCC GGTCTGCCCC TACCGTGGCC AGTGCCGCTC CTCCCTCCGT
CCACGGGCGA CCCTCCGCTT TCGCCCTTAC ACGAAGAGGA TGTGCAATGG CTGTGATGAC
CCCCCGCCGG GAGCGCTCTT CCCTGCTCTC CCGAGCTCTG CAAGTGACGG CTGCGGCTGC
CACAGCGCTT GTGACCGCGG TCAGCCTGGC CGCCCCCGCT CATGCCGCCA ACCCCTACGA
GCGCGGCCCC AACCCGACCG ACGCCCTGCT CGAAGCCAGC AGCGGCCCCT TCTCCGTCAG
CGAGGAGAAC GTCTCCCGGT TGAGCGCCAG CGGCTTCGGC GGCGGCACCA TCTACTACCC
GCGGGAGAAC AACACCTACG GTGCGGTGGC GATCTCCCCC GGCTACACCG GCACTGAGGC
TTCCATCGCC TGGCTGGGCG AGCGCATCGC CTCCCACGGC TTCGTCGTCA TCACCATCGA
CACCATCACC ACCCTCGACC AGCCGGACAG CCGGGCAGAG CAGCTCAACG CCGCGCTGAA
CCACATGATC AACCGGGCGT CCTCCACGGT GCGCAGCCGG ATCGACAGCA GCCGACTCGC
GGTCATGGGC CACTCCATGG GCGGCGGCGG CACCCTGCGT CTGGCCTCCC AGCGTCCCGA
CCTGAAGGCC GCCATCCCGC TCACCCCGTG GCACCTCAAC AAGAACTGGA GCAGCGTCAC
CGTGCCGACG CTGATCATCG GGGCCGACCT CGACACAATC GCGCCGGTCG CCACGCACGC
GAAACCGTTC TACAACAGCC TGCCGAGCTC CATCAGCAAG GCCTACCTGG AGCTGGACGG
CGCAACCCAC TTCGCCCCGA ACATCCCCAA CAAGATCATC GGCAAGTACA GCGTCGCCTG
GCTCAAGCGG TTCGTCGACA ACGACACCCG CTACACCCAG TTCCTCTGCC CCGGACCGCG
CGACGGACTC TTCGGCGAGG TCGAAGAGTA CCGCTCCACC TGCCCGTTCT AGGAAGAGAA
CACGACGAGT CTTTCCTCCC CATTCTTTCG GTGGCGGTCA CTGCGGTGGC CGCCACCGGC
CGTTTTGTCC CCCCTTTTCA TTCGAAAAAT GCGACAAACC ACCCTTTTTG CCCCATCGCA
CCCCCGATAC CGAACGAAGT TCGGGTACAA CACTGGTGGT TTTACGGATG CATGATCACT
GTGACTTGCC CCATAGTGGC AACGCAGTCG AGATAAGGAG CGCATAAACC CCAAACCTCC
TTACCTCCGC CTGCTGAGCG GCTCGTTGAC CGCACGTGGC CGGGCACACC GGCTTCGCCT
ACCGGTCGCA CGCGCCGTGC CTTCTCCACC CCCGCGGCGG AAAGGCCCGG CGCTCGCGGG
CTGGACCGTT CGGACCCCAC GCGAACGGCC CGGAACCCAT GGCACCCCCG CGTCCGGGAG
GCAAGCGCCG CGTGCCTACC GCCAACGGCG CCGCTCACCT CCAGTGGCGA GGCGGGAGTC
CGGGTCCACG TCCATGCATG CCCCCGCATG CGGCGCGGCC CGGCCCTGCA CAGAACCGAA
GAGGACGTGC AATGGCTGTG ATGACCCCCC GCCGGGAGCG CTCTTCCCTG CTCTCCCGGG
CACTGCGCTT CACCGCCGCG GCTGCCACAG CGCTTGTGAC CGCGGTCAGC CTGGCCGCCC
CCGCTCATGC CGCCAACCCC TACGAGCGCG GCCCCAACCC GACCGACGCC CTGCTCGAAG
CCCGCAGCGG CCCCTTCTCC GTGAGTGAAG AACGGGCCTC CCGCTTCGGT GCTGACGGTT
TCGGCGGCGG CACCATCTAC TACCCGCGGG AGAACAACAC CTACGGTGCC GTGGCGATCT
CCCCCGGCTA CACCGGCACC CAGGCCTCTG TCGCCTGGCT GGGCAAGCGC ATCGCCTCCC
ACGGCTTCGT CGTCATCACC ATCGACACCA ACACCACCCT CGACCAGCCG GACAGCCGGG
CCCGCCAGCT CAACGCCGCG CTGGACTACA TGATCAACGA CGCCTCGTCC GCGGTGCGCA
GCCGGATCGA CAGCAGCCGA CTGGCGGTCA TGGGCCACTC CATGGGCGGC GGCGGCAGCC
TGCGTCTGGC CTCCCAGCGT CCCGACCTGA AGGCCGCCAT CCCGCTCACC CCGTGGCACC
TCAACAAGAA CTGGAGCAGT GTGCGGGTTC CCACCCTCAT CATCGGTGCT GACCTGGACA
CCATCGCTCC GGTCCTCACC CACGCCCGGC CCTTCTACAA CAGCCTCCCG ACCTCGATCA
GCAAGGCCTA CCTGGAGCTG GACGGCGCAA CCCACTTCGC CCCGAACATC CCCAACAAGA
TCATCGGCAA GTACAGCGTC GCCTGGCTCA AGCGGTTCGT CGACAACGAC ACCCGCTACA
CCCAGTTCCT CTGCCCCGGA CCGCGCGACG GACTCTTCGG CGAGGTCGAA GAGTACCGCT
CCACCTGCCC CTTCTAGGCG GTAGGGTCCC GCAGCGAGTC AGCAAGATCT CCTTCCCGGT
GGTTGATACT G
or the complementary strand thereof,
(b) DNA sequences that hybridise under stringent conditions to protein-encoding regions of a DNA sequence according to (a) or to fragments of such a DNA sequence,
(c) DNA sequences that hybridise to DNA sequences according to (a) or (b) owing to degeneracy of the genetic code,
(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

2. A DNA sequence that comprises a DNA sequence selected from among:
(a) the following DNA sequence:
ATGGCTGTGA TGACCCCCCG CCGGGAGCGC TCTTCCCTGC TCTCCCGAGC TCTGCAAGTG
ACGGCTGCGG CTGCCACAGC GCTTGTGACC GCGGTCAGCC TGGCCGCCCC CGCTCATGCC
GCCAACCCCT ACGAGCGCGG CCCCAACCCG ACCGACGCCC TGCTCGAAGC CAGCAGCGGC
CCCTTCTCCG TCAGCGAGGA GAACGTCTCC CGGTTGAGCG CCAGCGGCTT CGGCGGCGGC
ACCATCTACT ACCCGCGGGA GAACAACACC TACGGTGCGG TGGCGATCTC CCCCGGCTAC
ACCGGCACTG AGGCTTCCAT CGCCTGGCTG GGCGAGCGCA TCGCCTCCCA CGGCTTCGTC
GTCATCACCA TCGACACCAT CACCACCCTC GACCAGCCGG ACAGCCGGGC AGAGCAGCTC
AACGCCGCGC TGAACCACAT GATCAACCGG GCGTCCTCCA CGGTGCGCAG CCGGATCGAC
AGCAGCCGAC TGGCGGTCAT GGGCCACTCC ATGGGCGGCG GCGGCACCCT GCGTCTGGCC
TCCCAGCGTC CCGACCTGAA GGCCGCCATC CCGCTCACCC CGTGGCACCT CAACAAGAAC
TGGAGCAGCG TCACCGTGCC GACGCTGATC ATCGGGGCCG ACCTCGACAC AATCGCGCCG
GTCGCCACGC ACGCGAAACC GTTCTACAAC AGCCTGCCGA GCTCCATCAG CAAGGCCTAC
CTGGAGCTGG ACGGCGCAAC CCACTTCGCC CCGAACATCC CCAACAAGAT CATCGGCAAG
TACAGCGTCG CCTGGCTCAA GCGGTTCGTC GACAACGACA CCCGCTACAC CCAGTTCCTC
TGCCCCGGAC CGCGCGACGG ACTCTTCGGC GAGGTCGAAG AGTACCGCTC CACCTGCCCG
TTC
or the complementary strand thereof,
(b) DNA sequences that hybridise under stringent conditions to protein-encoding regions of a DNA sequence according to (a) or to fragments of such a DNA sequence,
(c) DNA sequences that hybridise to DNA sequences according to (a) or (b) owing to degeneracy of the genetic code,
(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

3. A DNA sequence that comprises a DNA sequence selected from among:
(a) the following DNA sequence:
ATGGCACCCC CGCGTCCGGG AGGCAAGCGC CGCGTGCCTA CCGCCAACGG CGCCGCTCAC
CTCCAGTGGC GAGGCGGGAG TCCGGGTCCA CGTCCATGCA TGCCCCCGCA TGCGGCGCGG
CCCGGCCCTG CACAGAACCG AAGAGGACGT GCAATGGCTG TGATGACCCC CCGCCGGGAG
CGCTCTTCCC TGCTCTCCCG GGCACTGCGC TTCACCGCCG CGGCTGCCAC AGCGCTTGTG
ACCGCGGTCA GCCTGGCCGC CCCCGCTCAT GCCGCCAACC CCTACGAGCG CGGCCCCAAC
CCGACCGACG CCCTGCTCGA AGCCCGCAGC GGCCCCTTCT CCGTGAGTGA AGAACCGGCC
TCCCGCTTCG GTGCTGACGG TTTCGGCGGC GGCACCATCT ACTACCCGCG GGAGAACAAC
ACCTACGGTG CCGTGGCGAT CTCCCCCGGC TACACCGGCA CCCAGGCCTC TGTCGCCTGG
CTGGGCAAGC GCATCGCCTC CCACGGCTTC GTCGTCATCA CCATCGACAC CAACACCACC
CTCGACCAGC CGGACAGCCG GGCCCGCCAG CTCAACGCCG CGCTGGACTA CATGATCAAC
GACGCCTCGT CCGCGGTGCG CAGCCGGATC GACAGCAGCC GACTGGCGGT CATGGGCCAC
TCCATGGGCG GCGGCGGCAG CCTGCGTCTG GCCTCCCAGC GTCCCGACCT GAAGGCCGCC
ATCCCGCTCA CCCCGTGGCA CCTCAACAAG AACTGGAGCA GTGTGCGGGT TCCCACCCTC
ATCATCGGTG CTGACCTGGA CACCATCGCT CCGGTCCTCA CCCACGCCCG GCCCTTCTAC
AACAGCCTCC CGACCTCGAT CAGCAAGGCC TACCTGGAGC TGGACGGCGC AACCCACTTC
GCCCCGAACA TCCCCAACAA GATCATCGGC AAGTACAGCG TCGCCTGGCT CAAGCGGTTC
GTCGACAACG ACACCCGCTA CACCCAGTTC CTCTGCCCCG GACCGCGCGA CGGACTCTTC
GGCGAGGTCG AAGAGTACCG CTCCACCTGC CCCTTC
or the complementary strand thereof,
(b) DNA sequences that hybridise under stringent conditions to protein-encoding regions of a DNA sequence according to (a) or to fragments of such a DNA sequence,
(c) DNA sequences that hybridise to DNA sequences according to (a) or (b) owing to degeneracy of the genetic code,
(d) allelic variations and mutations produced by substitution, insertion or deletion of single or several nucleotides or inversion of single or several nucleotide partial sequences of the DNA sequences according to (a) to (c), the variations and mutants coding for isofunctional expression products.

4. A recombinant expression vector containing a DNA sequence according to any one of claims 1 to 3.

5. A prokaryotic or eukaryotic cell that has been transformed or transfected with a DNA sequence according to any one of claims 1 to 3 or with a recombinant expression vector according to claim 4.

6. An expression product or a partial expression product of a DNA sequence according to any one of claims 1 to 3 or of a recombinant expression vector according to claim 4.

7. A process for the production of an expression product or a partial expression product according to claim 6, in which a cell according to claim 5 is cultured in a suitable culture medium and the expression product or partial expression product is isolated from the cells and/or the culture medium.

8. An amino acid sequence that comprises an amino acid sequence selected from among
(a) the following amino acid sequence:
MAVMTPRRER SSLLSRALQV TAAAATALVT AVSLAAPAHA ANPYERGPNP TDALLEASSG
PFSVSEENVS RLSASGFGGG TIYYPRENNT YGAVAISPGY TGTEASIAWL GERIASHGFV
VITIDTITTL DQPDSRAEQL NAALNHMINR ASSTVRSRID SSRLAVMGHS MGGGGTLRLA
SQRPDLKAAI PLTPWHLNKN WSSVTVPTLI IGADLDTIAP VATHAKPFYN SLPSSISKAY
LELDGATHFA PNIPNKIIGK YSVAWLKRFV DNDTRYTQFL CPGPRDGLFG EVEEYRSTCP
F
(b) an amino acid sequence that is an allelic isofunctional variant of the amino acid sequence defined in (a),
(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,
(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

9. An amino acid sequence that comprises an amino acid sequence selected from among
(a) the following amino acid sequence:
MAPPRPGGKR RVPTANGAAH LQWRGGSPGP RPCMPPHAAR PGPAQNRRGR AMAVMTPRRE
RSSLLSRALR FTAAAATALV TAVSLAAPAH AANPYERGPN PTDALLEARS GPFSVSEERA
SRFGADGFGG GTTYYPRENN TYGAVAISPG YTGTQASVAW LGKRIASHGF VVITIDTNTT
LDQPDSRARQ LNAALDYMIN DASSAVRSRI DSSRLAVMGH SMGGGGSLRL ASQRPDLKAA
IPLTPWHLNK NWSSVRVPTL IIGADLDTTA PVLTHARPFY NSLPTSISKA YLELDGATHF
APNIPNKIIG KYSVAWLKRF VDNDTRYTQF LCPGPRDGLF GEVEEYRSTC PF
(b) an amino acid sequence that is an allelic isofunctional variant of the amino acid sequence defined in (a),
(c) a mutant of the amino acid sequence defined in (a) or (b), in which one or more amino acid residue(s) has/have been deleted and/or replaced and/or inserted and or one or more amino acid partial sequence(s) has/have been inverted, and that is isofunctional,
(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

10. An amino acid sequence that comprises an amino acid sequence selected from among
(a) the following amino acid sequence:
AANPYERGPN PTDALLEASS GPFSVSEENV SRLSASGFGG GTTYYPRENN TYGAVAISPG
YTGTEASIAW LGERIASHGF VVITIDTITT LDQPDSRAEQ LNAALNHMIN RASSTVRSRI
DSSRLAVMGH SMGGGGTLRL ASQRPDLKAA IPLTPWHLNK NWSSVTVPTL IIGADLDTIA
PVATHAKPFY NSLPSSISKA YLELDGATHF APNIPNKIIG KYSVAWLKRF VDNDTRYTQF
LCPGPRDGLF GEVEEYRSTC PF
(b) an amino acid sequence that is an allelic isofunctional variant of the amino acid sequence defined in (a),
(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,
(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

11. An amino acid sequence that comprises an amino acid sequence selected from among
(a) the following amino acid sequence:
AANPYERGPN PTDALLEARS GPFSVSEERA SRFGADGFGG GTIYYPRENN TYGAVAISPG
YTGTQASVAW LGKRIASHGF VVITIDTNTT LDQPDSRARQ LNAALDYMIN DASSAVRSRI
DSSRLAVMGH SMGGGGSLRL ASQRPDLKAA IPLTPWHLNK NWSSVRVPTL IIGADLDTIA
PVLTHARPFY NSLPTSISKA YLELDGATHF APNIPNKIIG KYSVAWLKRF VDNDTRYTQF
LCPGPRDGLF GEVEEYRSTC PF
(b) an amino acid sequence that is an allelic isofunctional variant of the amino acid sequence defined in (a),
(c) a mutant of the amino acid sequence defined in (a) or (b), in which one or more amino acid residue(s) has/have been deleted and/or replaced and/or inserted and/or one or more amino acid partial sequence(s) has/have been inverted, and that is isofunctional,
(d) an amino acid sequence having an amino acid sequence fused to the C-terminus and/or N-terminus thereof, wherein the resulting fusion protein is isofunctional and/or the additional C-terminal or N-terminal amino acid sequences can readily be removed.

12. A polyclonal or monoclonal antibody to an expression product or partial expression product according to claim 6 or to an amino acid sequence according to any one of claims 8 to 11.

13. The use of the cells according to claim 5, of the expression or partial expression products according to claim 6 or of the amino acid sequences according to any one of claims 8 to 11 for the biological degradation of polymers containing ester groups.

14. The use according to claim 13, wherein the polymers are aliphatic-aromatic polyesters.
